(19) United States
(12) Patent Application Publication
(10) Pub. No.: US 2022/0223253 A1
(43) Pub. Date: Jul. 14, 2022

(54) FUNCTION RECOVERY TRAINING SYSTEM, FUNCTION RECOVERY TRAINING DEVICE AND PROGRAM

(71) Applicant: Sumitomo Dainippon Pharma Co., Ltd., Osaka (JP)

(72) Inventor: Mari HIRAOKA, Koganei-shi, Tokyo (JP)

(73) Assignee: Sumitomo Dainippon Pharma Co., Ltd., Osaka (JP)

(21) Appl. No.: 17/609,486

(22) PCT Filed: May 1, 2020
(86) PCT No.: PCT/JP2020/018411

§ 371 Date: Nov. 8, 2021

(30) Foreign Application Priority Data
May 9, 2019 (JP) 2019-088811

(51) Int. Cl. G16H 20/30; G06T 7/00; G16H 50/20; A61B 3/14

(52) U.S. Cl. CPC G16H 20/30; G06T 7/0012; G06T 2207/30041; A61B 3/14; G16H 50/20

(57) ABSTRACT

There are provided a function recovery training system, a function recovery training device and a program making it possible to, by quantitatively grasping states of a plurality of biological functions of a subject (specifically, a plurality of functions related to vision including relationships with the brain and the optic nerve), easily, efficiently and effectively perform recovery training for the biological functions of the subject from a composite point of view. There is provided a function recovery training system 10 for performing training to recover biological functions of a subject, the function recovery training system including: evaluation means 11; program generation means 12; and storage means 13; wherein the storage means 13 holds: a standard program 101 capable of evaluating states of the functions to be evaluation criteria based on a plurality of function evaluation items; and evaluation-item-specific programs 102 making it possible to perform training individually for each of the plurality of function evaluation items; the evaluation means 11 executes the standard program 101 and evaluates criterial states of the functions based on a response from the subject; the program generation means 12 selects any of the evaluation-item-specific programs 102 that is for at least one of the plurality of function evaluation items based on the evaluated criterial states to generate a training program 103; and the evaluation means 11 executes the training program 103 and evaluates recovery states of the functions based on a response from the subject.

### TECHNICAL FIELD

The present invention relates to a function recovery training system, a function recovery training device and a program.

### BACKGROUND ART

Conventionally, techniques for performing measurement or training about states of visual functions (for example, visual acuity) or visual dysfunctions (for example, diplopia, strabismus and the like) have been known (see, for example, Patent Literatures 1 and 2).

These techniques are intended to, for a certain particular visual function or visual dysfunction, individually grasp (measure) states thereof and give training, and provide methods for measurement and training corresponding to each of visual functions and abnormalities (symptoms) thereof mainly by stimulating ocular muscles.

By the way, the optic nerve, cerebral nerves or the brain other than 25 the optic nerve, or linkage parts among them are complicatedly related to vision, and a visual dysfunction is not limited merely to an abnormality of an ophthalmic function (for example, a function mainly by work of ocular muscles and eyeballs, a function demonstrated by parts in a so-called orbit) but may be caused by an abnormality or dysfunction of the brain, a cerebral 30 nerve related to vision (the optic nerve) or a linkage part among them.

For example, especially when a visual dysfunction is caused by an acquired abnormality of brain functions, effective function recovery cannot be expected even if only ophthalmic function recovery training (for example, training that stimulates ocular muscles) is performed.

On the other hand, for example, by stimulating a part of the brain or the optic nerve via behavior of “seeing an aim object”, a part of the brain or the optic nerve where an abnormality has occurred may be recovered, or a visual function that has been insufficient (therefore, brain and optic nerve functions) may be recovered by another part replacing with the abnormal part.

### CITATION LIST

### Patent Literature

Patent Literature 1: Japanese Patent Laid-Open No. 9-51927
Patent Literature 2: Japanese Patent Laid-Open No. 2005-524432

### SUMMARY OF INVENTION

### Technical Problem

However, in the case of judging a visual dysfunction, excluding obviously ophthalmic dysfunctions (for example, a dysfunction only because of an ocular muscle abnormality and the like), it is very difficult to, for the 25 cause of the dysfunction or a part where the abnormality has occurred, individually separate the brain, the optic nerve, the ocular muscles or linkage parts among them to identify each alone, and it can be said that it is impossible.

Further, a lot of effort is required to accurately identify an abnormal part and the cause of a dysfunction, including relationships with the brain and the optic nerve.

Furthermore, for example, surgical treatment (such as a surgery), function recovery training and the like are conceivable in order to recover visual functions. However, the surgical treatment is a heavy burden on a subject (a patient; hereinafter the same), and a training method varies according to the subject's symptoms and a judgment by a medical worker (a doctor or the like). Therefore, as for a viewpoint that anyone easily performs efficient and effective training, it has not been sufficiently achieved.

Especially, in the case of a visual dysfunction in which the brain and the optic nerve are involved, it has a higher priority, for the subject, to become able to live his daily life without trouble by recovering functions about the vision and functions of the optic nerve and the brain by training than to accurately identify the part and cause of the abnormality, and, furthermore, it is further desired that the training can be easily performed and is effective.

### Solution to Problem

In view of the above situation, the present invention provides a function recovery training system, a function recovery training device and a program making it possible to, by quantitatively grasping states of a plurality of biological functions of a subject (specifically, a plurality of functions related to vision including relationships with the brain and the optic nerve), easily, efficiently and effectively perform recovery training for the biological functions of the subject from a composite point of view.

That is, the present invention is a function recovery training system for performing training to recover biological functions of a subject, the function recovery training system including a terminal device, the terminal device including: evaluation means; program generation means; storage means; input means; and output means, wherein the storage means holds: a standard program capable of evaluating states of the functions to be evaluation criteria based on a plurality of function evaluation items; and evaluation-item-specific programs making it possible to perform training individually for each of the plurality of function evaluation items; wherein the evaluation means outputs a predetermined target to the output means by execution of the standard program, accepts a response to the target from the subject via the input means, and evaluates criterial states of the functions based on the response; wherein the program generation means selects any of the evaluation-item-specific programs that is for at least one of the plurality of function evaluation items based on the evaluated criterial states to generate a training program; and wherein the evaluation means outputs a predetermined target to the output means by execution of the training program, accepts a response to the target from the subject via the input means, and evaluates recovery states of the functions based on the response.

Further, the present invention is a function recovery training device for performing training to recover biological functions of a subject, the function recovery training device including: evaluation means; program generation means; storage means; input means; and output means, wherein the storage means is means for holding: a standard program capable of evaluating states of the functions to be evaluation criteria based on a plurality of function evaluation items; and evaluation-item-specific programs making it possible to perform training individually for each of the plurality of function evaluation items; wherein the evaluation means is means for outputting a predetermined target to the output means by execution of the standard program, accepting a response to the target from the subject via the input means, evaluating criterial states of the functions based on the response; outputting a predetermined target to the output means by execution of a training program, accepting a response to the target from the subject via the input means, and evaluating recovery states of the functions based on the response; and wherein the program generation means selects any of the evaluation-item-specific programs that is for at least one of the plurality of function evaluation items based on the evaluated criterial states to generate the training program.

Further, the present invention is a program including a standard program and evaluation-item-specific programs, the program causing a computer to execute a recovery training process for biological functions, wherein the standard program is configured to be capable of evaluating states of the functions to be evaluation criteria based on a plurality of function evaluation items; wherein the evaluation-item-specific programs are configured to make it possible to individually perform training for the plurality of function evaluation items, respectively; and wherein the evaluation-item-specific programs cause the computer to execute the steps of: executing the standard program to output a predetermined target to a subject; accepting a response to the target from the subject; evaluating criterial states of the functions based on the response; selecting any of the evaluation-item-specific programs that is for at least one of the plurality of function evaluation items based on the evaluated criterial states to generate a training program; executing the training program to output a predetermined target to the subject; accepting a response to the target from the subject; and evaluating recovery states of the functions based on the response.

### Advantageous Effects of Invention

According to the present invention, it is possible to achieve an excellent effect that there are provided a function recovery training system, a function recovery training device and a program making it possible to, by quantitatively grasping states of a plurality of biological functions of a subject (specifically, a plurality of functions related to the vision including relationships with the brain and the optic nerve), easily, efficiently and effectively perform recovery training for the biological functions (functions related to the vision, functions of the optic nerve and the brain, and the like) of the subject from a composite point of view.

### BRIEF DESCRIPTION OF DRAWINGS

FIG. 1 is a block diagram showing an example of a configuration of a function recovery training system according to an embodiment of the present invention.

FIG. 2 is a table showing an example of function evaluation items of the function recovery training system according to the embodiment of the present invention.

FIG. 3 is a flowchart showing an example of a process of the function recovery training system according to the embodiment of the present invention.

FIG. 4 is a diagram showing an example of the function recovery training system according to the embodiment of the present invention, wherein (A) is an overview diagram illustrating a standard program, and (B) to (G) are overview diagrams showing examples of evaluations of functions.

FIG. 5 is a diagram showing an example of the function recovery training system according to the embodiment of the present invention, wherein (A) is a diagram showing an example of display of evaluation results, and (B) is an overview diagram illustrating a training program.

### DESCRIPTION OF EMBODIMENT

An embodiment of the present invention will be explained with reference to accompanying drawings.

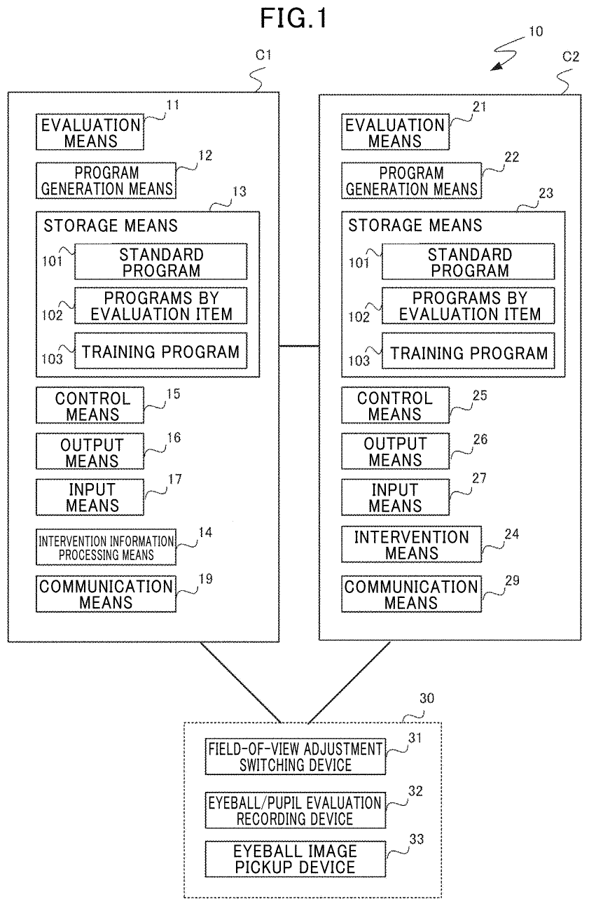

FIG. 1 is a schematic block diagram showing an example of a configuration of a function recovery training system 10 according to the embodiment of the present invention.

The function recovery training system 10 of the present embodiment is for performing training so that biological functions of a subject can be recovered to a predetermined level.

Here, the “biological functions” in the present embodiment are, for example, “functions related to vision (seeing)”. Relationships between the vision and the optic nerve, cerebral nerves or the brain other than the optic nerve, or linkage parts among them are complicated, and some portions are not strictly clearly classified. In the present embodiment, explanation will be made with definitions below as an example for convenience of the explanation.

For example, the “functions related to vision (seeing)” are mainly visual functions (including functions of the optic nerve, cerebral nerves or the brain other than the optic nerve, and linkage parts among them that are related to the vision).

Further, it is assumed that the visual functions of the present embodiment include ophthalmic functions, neuro-ophthalmic functions and encephalo-ophthalmic functions.

The ophthalmic functions are functions of capturing (inputting) external information, and refer to functions demonstrated by a part in the orbit (for example, functions of opening eyes by raising eyelids, aiming eyeballs at an aim object to see the object, bringing the aim object into focus and the like), and include functions of visual acuity/color vision for clearly seeing what the subject wants to see, refraction/adjustment for bringing what the subject wants to see into focus, convergence/binocular vision for grasping the perspective of what the subject wants to see, vision fixation/eyeball movement for correctly aiming the line of sight at what the subject wants to see, and the like.

The neuro-ophthalmic functions are functions in which the brain (mainly the diencephalon, the cerebellum and the midbrain) is involved, and refer to a function of performing information processing such as using ocular muscles of both eyes to adjust the lines of sight toward an aim object, and reading a distance from a difference between the visual angles thereof or stereoscopically seeing an object, and/or a function of processing inputted information to correctly judge what the subject wants to see (and communicating (outputting) the processed information to the motor function).

The neuro-ophthalmic functions include, for example, gaze, pursuit (following), near reaction, distance discrimination (stereopsis), short-term remembrance, reaction to an aim object that has started to move, and the like.

The encephalo-ophthalmic functions refer to function that are closely related to the ophthalmic functions and/or the neuro-ophthalmic functions among brain functions (higher brain functions) and include functions in which the cerebrum is involved, such as (long-term) remembrance, judgment and thinking.

The function recovery training system 10 includes a terminal device (a subject terminal device) C1 that the subject is provided with. The subject terminal device C1 is, for example, a mobile information terminal (for example, a smartphone, a tablet terminal, a mobile phone, a PDA (personal digital assistant) or the like), a personal computer (PC) or the like.

The subject terminal device C1 has evaluation means 11, program generation means 12, storage means 13, intervention information processing means 14, control means 15, output means 16, input means 17, communication means 19 and the like.

The evaluation means 11 is means (a function) realized by a part of a function recovery training software program (application software) that is introduced into the subject terminal device C1 and executed, and is, for example, means for at least performing predetermined output (for example, display) to the subject, accepting a response (an input) of the subject, and performing predetermined arithmetic operation to evaluate the response.

Specifically, the evaluation means 11 evaluates criterial states (to be described later) of functions of the subject by executing at least a standard program 101 and evaluates recovery states (to be described later) of the functions of the subject by executing a training program 103.

The storage means 13 holds the standard program 101, evaluation-item-specific programs 102, the training program 103 and the like.

Details of each of the programs will be described later. The standard program 101 is, for example, a program provided for a plurality of subjects in common and is configured to be capable evaluating states of functions of each subject to be evaluation criteria based on a plurality of function evaluation items. The plurality of function evaluation items are evaluation items for the ophthalmic functions, the neuro-ophthalmic functions and the encephalo-ophthalmic functions in this example.

By execution of the standard program 101, the evaluation means 11 outputs a predetermined target to the output means 16. When the subject responds thereto, the evaluation means 11 makes judgments (evaluations) about the plurality of function evaluation items by appropriately performing arithmetic processing and the like in response to the response to evaluate criterial states of the functions of the subject. The function evaluation items will be described later.

Further, by execution of the training program 103, the evaluation means 11 outputs a predetermined target to the output means 16. When the subject responds thereto, the evaluation means 11 makes judgments (evaluations) about one or more function evaluation items by appropriately performing arithmetic processing and the like in response to the response to evaluate the recovery states of the functions of the subject. Evaluation results of the evaluation means 11 (the criterial states and the recovery states) are stored and accumulated in the storage means 13.

The evaluation-item-specific programs 102 are configured to make it possible to individually perform training for a plurality of function evaluation items, respectively. The plurality of function evaluation items of the evaluation-item-specific programs 102 are also evaluation items for the ophthalmic functions, the neuro-ophthalmic functions and the encephalo-ophthalmic functions (details will be described later). The evaluation-item-specific programs 102 are prepared so that one or more of them are selected by the program generation means 12 described later to generate a training program 103.

Here, for example, each of the evaluation-item-specific programs 102 is configured such that, when the program by evaluation item 102 is executed, and the subject responds thereto, a burden (a levels or a difficulty degree) on the subject is different from that of a corresponding function evaluation item of the standard program 101. The burden on the subject is, for example, time for the subject to respond (for example, the number of questions and the like), stimulation on the subject (stimulation to cause the function to work), the number of repetitions of the subject's response, the difficulty degree of the response and the like. In this case, not only a program with a burden heavier than that of the standard program but also a program with a lighter burden is possible. The burdens of both programs on the subject may be equal.

The training program 103 is generated by the program generation means 12 and is configured to make it possible to perform training for at least one function evaluation item among the plurality of function evaluation items described above. The training program 103 is also configured such that, when the training program 103 is executed, and the subject responds thereto, a burden on the subject is different from that of the standard program 101. In this case, not only a program with a burden heavier than that of the standard program but also a program with a lighter burden is possible. The burdens of both programs on the subject may be equal.

Based on evaluation results of the standard program by the evaluation means 11, that is, evaluation results of the states of the functions of the subject who has responded to the standard program (the criterial states), the program generation means 12 selects a program by evaluation item for at least one function evaluation item among the plurality of function evaluation items and, in the case of a plurality of evaluation-item-specific programs, combines them to generate the training program.

The control means 15 is configured with a CPU, a RAM, a ROM and the like and executes various kinds of controls including controls of the evaluation means 11 and the program generation means 12. The CPU is a so-called central processing unit, and various kinds of programs are executed to realize various kinds of functions. The RAM is used as a working area of the CPU. The ROM stores a basic OS and programs to be executed by the CPU. Further, the control means 15 includes means for estimating and reading positions of an aim object (a target), an indicator (a pointer or a cursor) on XY coordinates taking a center of output means 16 as an origin, and measuring a distance between the aim object and an indicator on the coordinates, and means for measuring times to start movement and moving speeds of the aim object (the target) and the indicator (the pointer or the cursor) by being combined with a clock function.

The output means 16 is means for outputting a predetermined target at least based on execution of the standard program 101 and the training program 103 and is, for example, display means (a display) for displaying a predetermined aim object (for example, a target or the like) to the subject. As the output means 16, voice output means (a speaker) may be provided in addition to the display means.

The input means 17 is means capable of inputting a response of the subject to a target (for example, an aim object) outputted by the output means 16 and is, for example, operation means (specifically, a keyboard, a mouse, a touch panel, a joystick, a button, a pointing device or the like) or voice input means (a microphone or the like).

The intervention information processing means 14 is means for accepting information from outside (for example, external devices 30 or a medical worker terminal device C2 (to be described later)) or an input by an operation of the input means 17 and the like (intervention information), and performing information processing (arithmetic operation, conversion and the like) required to reflect the information on evaluations by the evaluation means 11 (evaluations of the criterial states and the recovery states) as necessary. If the intervention information processed by the intervention information processing means 14 exists in the storage means 13 or the like, the evaluation means 11 reflects the intervention information to make evaluations at the time of evaluating the criterial states and the recovery states.

The intervention information is, for example, evaluation (measurement and judgment) results by a known external device 30 or method, and is, for example, measurement (test) results by a visual acuity test device, a color vision test device and a corneal shape/refraction analysis device, a diplopia judgment result using a HESS chart or the like, or evaluation results of a Hasegawa simple intelligence evaluation scale, which is a simple evaluation scale for dementia, and the like. Or alternatively, the intervention information is information transmitted from the medical worker terminal device C2.

The communication means 19 is means for performing transmission/reception of data and the like to/from external devices (the external devices 30, the medical worker terminal device C2, other subject terminal devices C1, and the like) via a network.

Further, the function recovery training system 10 of the present embodiment is provided with the terminal device (the medical worker terminal device) C2 used by a medical worker (or a function recovery training specialist or the like) that is different from the subject terminal device C1. The medical worker terminal device C2 is, for example, a mobile information terminal (for example, a smartphone, a tablet terminal, a mobile phone, a PDA (personal digital assistant) or the like), a personal computer (PC) or the like, and connects to the subject terminal device C1 via a communication line (wired, wireless, a telephone line, or a communication network such as the Internet).

The medical worker terminal device C2 is configured similarly to the subject terminal device C1. That is, the medical worker terminal device C2 has evaluation means 21, program generation means 22, storage means 23, control means 25, output means 26, input means 27, communication means 29 and the like. Since the evaluation means 21, the program generation means 22, the storage means 23, the control means 25, the output means 26, the input means 27 and the communication means 29 are configured similarly to those means of the subject terminal device C1, explanation will be omitted.

The standard program 101, the evaluation-item-specific programs 102 and the training program 103 are similarly introduced into the medical worker terminal device C2.

Further, the medical worker terminal device C2 is provided with intervention means 24. For example, the intervention means 24 can perform intervention processing such as correction and change for the standard program 101, the evaluation-item-specific programs 102, the training program 103 and the like of the subject terminal device C1 and can transmit intervention information (for example, an altered program, data and the like) obtained as a result of the intervention processing to (the intervention information processing means 14 of) the subject terminal device C1 via the communication means 29.

Further, data of evaluation results (the criterial states and recovery states of the subject) in the subject terminal device C1 can be transmitted to the medical worker terminal device C2 via the communication means 19. That is, the training program 103 may be generated by the function recovery training system 10 in the subject terminal device C1 or may be generated by the function recovery training system 10 in the medical worker terminal device C2 and transmitted to the function recovery training system 10 in the subject terminal device C1 after the intervention processing (for example, alteration of a program, addition of data, and the like) by the medical worker is performed as necessary.

Furthermore, the function recovery training system 10 of the present embodiment may be provided with known external devices (means) 30. The external devices are, for example, a field-of-view adjustment switching device (means) 31, an eyeball/pupil evaluation recording device recording device (means) 32, an eyeball image pickup device (means) 33 and the like.

The field-of-view adjustment switching device (means) 31 is means for quantitatively evaluating neuro-ophthalmic functions and is adapted to make it possible to see a visual target on the display means 16 with monocular vision or binocular vision, that is, with one or both of right and left eyes, and is a device to block the field of view or make the visual target invisible by an ophthalmic function or a neuro-ophthalmic function to be evaluated and recovered. The field-of-view adjustment switching device 31 is, for example, red-blue glasses the left and right lenses of which are different in color (with red and blue lenses) or a screen to be placed on glasses or a face (a wearable device) according to purposes, which may be of a type of blocking left and right fields of view by an electronic mechanism. A switching signal of the field-of-view adjustment switching device 31 is transmitted from the subject terminal device C1 and/or the medical worker terminal device C2. Further, information of the switching signal and clock information at that time are transmitted to the subject terminal device C1 and/or the medical worker terminal device C2 together with evaluation results of the standard program 101 and the training program 103.

The eyeball/pupil evaluation recording device (means) 32 evaluates and records movements of the subject's eyeballs/pupils and transmits results to the subject terminal device C1 and/or the medical worker terminal device C2. The eyeball/pupil evaluation recording device 32 is installed to measure and record orientations (angles) of eyeballs of left and right eyes and the sizes of pupils (constricted/dilated diameters) from the left and right of glasses of the field-of-view adjustment switching device and is started especially at the time of evaluating near reaction of the neuro-ophthalmic functions. The visual angle as an ophthalmic function and the near reaction as a neuro-ophthalmic function of the subject may be individually measured and evaluated separately (in advance), and only results thereof may be inputted to the function recovery training system 10 via the intervention information processing means 14 as intervention information.

The eyeball image pickup device (means) 33 is, for example, a camera to read the diameters of pupils, convergence and divergence of eyeballs at the time of near reaction. Results of the reading (images or video) are transmitted to the subject terminal device C1 and/or the medical worker terminal device C2. When the subject terminal device C1 is a mobile terminal (for example, a smartphone), the eyeball image pickup device (means) 33 is a camera provided in the mobile terminal. In addition thereto, means for capturing video and voice may be provided.

The eyeball image pickup device 33 judges, for example, behavior of the subject, especially a distance of the subject from the display means 16, and/or a distance between eyes, and/or which of one eye and both eyes a test (training) is to be performed with, and which eye is closed in the former case, and/or orthogonality of the visual axis relative to rotation of the display means 16 around the visual axis and/or the surface of the display means 16, items of information about eyes, activity of the eyes during measurement, and the like, and causes results of the judgments to be incorporated into evaluations. Further, for example, when the subject terminal device C1 is a mobile terminal (for example, a smartphone), a state of the subject terminal device C1, the operation and attitude of the device (for example, via a gyro sensor or the like), surrounding brightness, time and the like may be acquired together to be used for the evaluations.

Further, the eyeball image pickup device 33 may be a fundus camera, an OCT (optical coherence tomography) device, an SLO (scanning laser ophthalmoscope) or the like. In this case, the eyeball image pickup device 33 acquires a picked-up ophthalmic image. As examples of the ophthalmic image, there are a fundus image picked up by the fundus camera, a tomographic image of the fundi picked up by OCT, an image of a retina or a choroid picked up by SLO, and the like. The eyeball image pickup device 33 may be provided with a storage portion, and an ophthalmic image may be stored in association with a photographed part and photographing time and the like, which are photographing information.

Furthermore, the eyeball image pickup device 33 may be a glasses-type wearable device.

When these external devices (the field-of-view adjustment switching means (device) 31, the eyeball/pupil evaluation recording means (device) 32 and the eyeball image pickup means (camera) 33 and the like) are bodies separate from the function recovery training system 10, the function recovery training system 10 is provided with synchronization means to synchronize outputs from the external devices with the output means (the display means) 16 of the function recovery training system 10. Further, the field-of-view adjustment switching means 31, the eyeball/pupil evaluation recording means 32 and the eyeball image pickup means 33 and the like may be provided integrally with the function recovery training system 10 (in the same case).

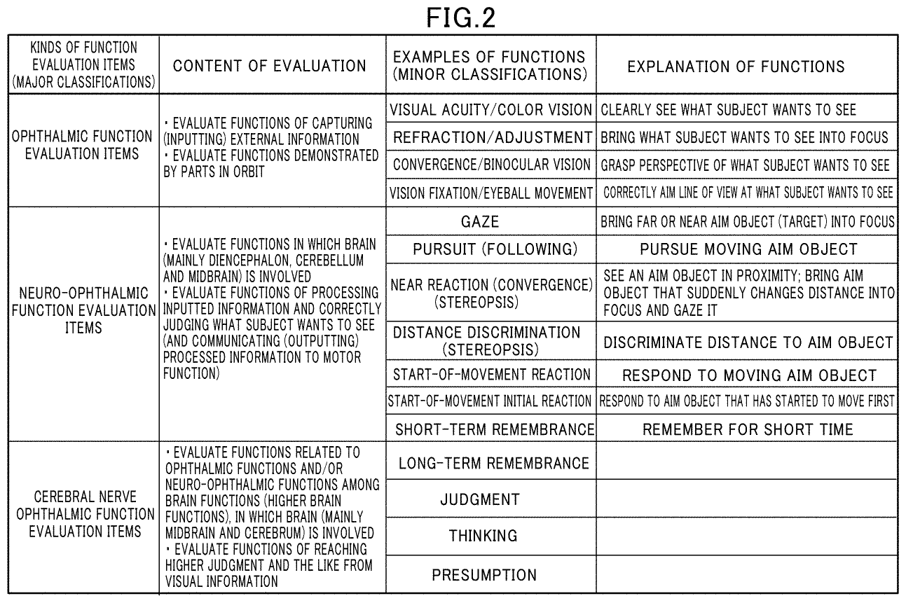

An example of the function evaluation items will be explained with reference to FIG. 2.

The function evaluation items of the present embodiment include, for example, evaluation items for ophthalmic functions (ophthalmic function evaluation items), evaluation items for neuro-ophthalmic functions (neuro-ophthalmic function evaluation items) and evaluation items for encephalo-ophthalmic functions (encephalo-ophthalmic function evaluation items).

Specifically, the ophthalmic function evaluation items are items of evaluating functions of capturing (inputting) external information or items of evaluating functions demonstrated by parts in the orbit (for example, functions of opening eyes by raising eyelids, aiming eyeballs at an aim object to see the object, bringing an aim object into focus and the like). As examples, items of evaluating a function of visual acuity/color vision (clearly seeing what the subject wants to see), a function of refraction/adjustment (bringing what the subject wants to see into focus), a function of convergence/binocular vision (grasping the perspective of what the subject wants to see), a function of vision fixation/eyeball movement (correctly aiming the line of sight at what the subject wants to see) and the like are included.

The neuro-ophthalmic function evaluation items are items of evaluating a function of performing information processing such as using ocular muscles of both eyes to adjust lines of sight toward an aim object, and reading a distance from a difference between visual angles thereof or stereoscopically seeing an object, and/or a function of processing inputted information and correctly judging what the subject wants to see (and communicating (outputting) the processed information to the motor function), the brain (mainly the diencephalon, the cerebellum and the midbrain) being involved in the functions. As examples, items of evaluating a function of gaze (bringing a far or near aim object (target) into focus) and a function of pursuit (following) (pursuing an aim object that is moving) are included as monocular vision functions. Further, items of evaluating a function of near reaction (convergence) (seeing an aim object in proximity; bringing an aim object that suddenly changes the distance into focus to gaze it), a function of distance discrimination (stereopsis) (discriminating a distance to an aim object), a function of start-of-movement reaction (reacting to an aim object that has started to move), a function of start-of-movement initial reaction (initially reacting to an aim object that has started to move), a function of short-term remembrance (about remembrance for a short time) and the like are included as binocular visions functions.

The cerebral nerve ophthalmic function evaluation items refer to evaluation items for functions in which the brain (mainly the midbrain and the cerebrum) is involved and which are closely related to the ophthalmic functions and/or the neuro-ophthalmic functions among brain functions (higher brain functions), the functions are functions of reaching a higher judgment and the like from visual information. Specifically, items of evaluating functions of long-term remembrance, judgment, thinking, presumption and the like are included.

Though the ophthalmic function evaluation items, the neuro-ophthalmic function evaluation items and the cerebral nerve ophthalmic function evaluation items are conveniently classified in FIG. 2, the functions of the items may be mutually correlated with one another. For example, the ophthalmic functions achieve an advanced function in close relationships and cooperation with parasympathetic nerves of the autonomic nervous system, and it is known that a higher central nervous system is also maintained by information stimulation brought about as a result thereof.

A specific example will be given. As for convergence, in the case of focusing the lines of sight of both eyes to an aim object in front of the eyes, the function can be said to be an ophthalmic function; but, in the case of bringing the aim object into focus to gaze it when the aim object has been moved back or forth, the function can be said to be a neuro-ophthalmic function because the brain (the midbrain) is also involved.

Further, in the case of stereopsis, it is thought that difference between images on the retinas of right and left eyes is processed in the brain, a solid is recognized by the images on both eyes being fused, and a depth is perceived. There may be a case where, when seeing a two-dimensional aim object, the object is three-dimensionally perceived due to psychological and remembrance (experiential) factors, and it can be said to correlate with the neuro-ophthalmic functions or the cerebral nerve ophthalmic functions.

In the present embodiment, such correlation relationships are held in the storage means 13 as correlation data, and the correlation data is used for evaluations of the criterial states and the recovery states by the evaluation means 11. For example, when evaluating the function of convergence, it is possible to make a quantitative evaluation for convergence as an ophthalmic function evaluation item. Therefore, in the case of evaluating convergence as a neuro-ophthalmic function evaluation item, it is possible to quantitatively evaluate convergence as a neuro-ophthalmic function in consideration of influence of the convergence that has been quantitatively evaluated as an ophthalmic function evaluation item (for example, including or excluding the influence). The correlation data may be data showing correlation beyond the classifications of functions such as the ophthalmic function evaluation items and the neuro-ophthalmic function evaluation items, or may be data, for example, showing correlation among a plurality of function evaluation items among the neuro-ophthalmic function items, such as gaze and pursuit.

Further, a function (function evaluation items) that is included in a plurality of kinds among the three kinds of functions of the ophthalmic functions, the neuro-ophthalmic functions and the cerebral nerve ophthalmic functions shown in FIG. 2 may be included. In addition to the functions illustrated in FIG. 2, functions other than those may be included. For example, if it is possible to evaluate diplopia, constriction and dilation of pupils, monocular vision, visual angles, the angle of visibility and the like, it is preferable.

As examples, a method used for a device for quantitatively and highly efficiently performing measurement using a known HESS chart (see Japanese Patent No. 3587468) can be used for evaluation of diplopia; and, for evaluations of constriction and dilation of pupils and convergence (rotation of eyeballs), a method used for a known device for quantitatively measuring functions of adjusting the constriction and dilation of pupils and the convergence (rotation of eyeballs) (see Japanese Patent Laid-Open No. 2002-17677) can be used.

As for the standard program 101, one continuous program is configured by making a combination so that at least any of the functions (minor classifications) of the ophthalmic function evaluation items (a major classification) as shown in FIG. 2, and at least any of the functions (minor classifications) of the neuro-ophthalmic function evaluation items (a major classification) can be evaluated. For example, the standard program 101 is configured so that at least evaluation of visual acuity and evaluation of gaze are compositely made in succession.

Further, if at least one function (a minor classification) is selected for each of the three function evaluation items of the ophthalmic function evaluation items, the neuro-ophthalmic function evaluation items and the cerebral nerve ophthalmic function evaluation items (major classifications), and the standard program 101 is configured so that the functions can be evaluated, it is more favorable. For example, if the standard program 101 is configured so that at least evaluation of refraction, evaluation of near reaction and evaluation of thinking are compositely made in succession, it is preferable.

Due to such a configuration, the subject compositely receives evaluations in succession for at least any of the functions (minor classifications) of the ophthalmic function evaluation items (a major classification) and at least any of the functions (minor classifications) of the neuro-ophthalmic function evaluation items (and the cerebral nerve ophthalmic function evaluation items) (a major classification) by responding to a series of outputs by execution of a certain one standard program 101. Results thereof become the criterial states of the subject.

Further, as for the evaluations in this case, by quantitatively grasping states of the functions, for example, using scores, points or the like and, if there are correlations among a plurality of function evaluation items (when correlation data can be used), considering the correlations, the evaluation (judgment) is made.

Then, based on the evaluation results (the criterial states), it is judged that a dysfunction has occurred, for a function evaluation item with a low evaluation; and, by appropriately selecting a program by evaluation item to recover the function to generate a training program 103, and causing the subject to respond to the training program 103, function recovery training for the subject is performed.

In the present embodiment, if there are correlations among a plurality of function evaluation items, evaluation is made in consideration of the correlations (correlation data). However, since the plurality of function evaluation items are (may be) closely related, it is not especially necessary to strictly identify a part that causes the dysfunction (for example, whether an ophthalmic function is insufficient or a neuro-ophthalmic function is insufficient, and the like).

That is, the function recovery training system 10 of the present embodiment is not intended to strictly identify a part that causes a dysfunction or the cause of the dysfunction but is intended to recover the function to a level at which the subject can spend his everyday life without trouble. That is, if evaluation of a function evaluation item is improved, it is thought that the function has been recovered, and it is possible to cause the subject to repeatedly execute a training program so that evaluation is improved.

Since there may be a case where there are correlations among a plurality of function evaluation items, it is recommended to appropriately add a program by evaluation item for a function with a good evaluation to the training program 103 in addition to a program by evaluation item for a function for which a low evaluation has been judged. This is because, if a good result is obtained for the function for which a low evaluation has been judged as a result, due to any of correlations among a plurality of function evaluation items, the function can be judged to have recovered.

That is, in the training program 103, in order that it becomes possible to make evaluations for evaluation items of functions (minor classifications) of at least any of the classifications of the ophthalmic function evaluation items, the neuro-ophthalmic function evaluation items and the cerebral nerve ophthalmic function evaluation items (the major classifications), preferably, of a plurality of kinds among the three kinds, one continuous program is configured by these evaluation items being combined. That is, by responding to a series of outputs by execution of a certain one training program 103, the subject receives training for any of the classifications of the ophthalmic function evaluation items, the neuro-ophthalmic function evaluation items or the cerebral nerve ophthalmic function evaluation items, preferably two or more kinds among them.

Evaluation by the training program 103 is quantitatively made, and, if there is correlation (correlation data) among function evaluation items, evaluation is made in consideration of the correlation, and the recovery states are judged.

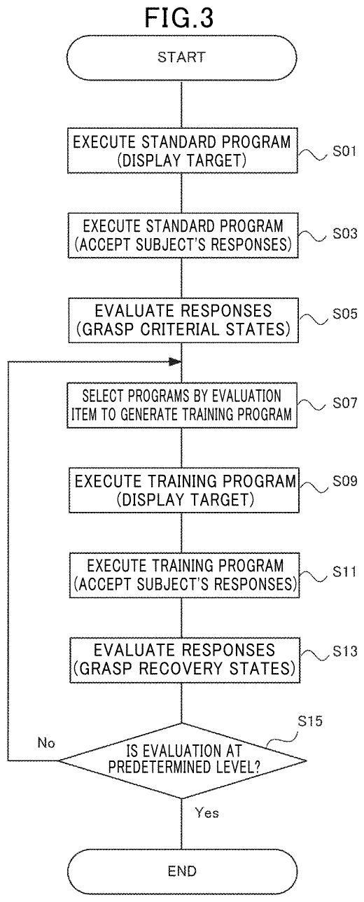

FIG. 3 is a flowchart showing an example of a flow of a process of the present system.

In the function recovery training system 10 of the present embodiment, first, the evaluation means 11 performs predetermined output (display of an aim object) on the output means 16 by execution of the standard program 101. Specifically, a moving target or the like is displayed on the display means 16, and the subject is prompted to make a response (step S01).

The standard program 101 appropriately combines outputs (displays) capable of successively and compositely evaluating the ophthalmic functions, the neuro-ophthalmic functions and the cerebral nerve ophthalmic functions to display the outputs in succession. For example, the display may be one in which a colored target is displayed first, and the subject is caused to answer the color. Next, the subject is caused to gaze the target for a predetermined time. Next, the target is moved, and the subject is caused to pursue the target. Next, the target is erased for a predetermined time, and the subject is caused to answer what kind of target the target was.

The evaluation means 11 accepts response inputs (responses to a plurality of function evaluation items) by the subject via the input means 17. For example, the response inputs are responses by operations of a touch panel (a mouse) or the like, voice inputs by voice input means (a microphone), and the like by the subject (step S03).

Then, based on the inputted responses, the evaluation means 11 evaluates the current states of the ophthalmic functions, the neuro-ophthalmic functions and the cerebral nerve ophthalmic functions of the subject, that is, states to be criteria (the criterial states) and extracts evaluation items thought to be required for the subject (step S05).

The program generation means 12 selects evaluation-item-specific programs making it possible to perform training individually for the evaluation items required for the subject, respectively, based on evaluation results of the evaluation means 11 (the criterial states). For the evaluations of the criterial states, correlation data of the ophthalmic function evaluation items, the neuro-ophthalmic function evaluation items and/or the cerebral nerve ophthalmic function evaluation items is also used.

The program generation means 12 selects, for example, a program making it possible to perform training, focusing on a dysfunction (an item the criterion of which has not been reached) to generate a training program 103 (step S07). Though only an item corresponding to a dysfunction may be extracted, it is desirable to select and combine a plurality of evaluation-item-specific programs not only for a single item but for items including those corresponding to normal functions and those corresponding to dysfunctions, because function evaluation items are often related to one another, and such relationships are not clear for some items.

The evaluation means 11 performs predetermined output (display of an aim object) on the output means 16 by execution of the generated training program 103. Specifically, a moving target or the like is displayed on the display means 16, and the subject is prompted to make a response (step S09). The display in this case is, for example, such display that the level of a burden on the subject is different from that of the standard program 101. For example, in the case of displaying a moving target or the like similar to that of the standard program 101 on the display means 16, the display is such that the movement speed is different from that of the standard program 101 (the movement speed is higher or lower).

The evaluation means 11 accepts response inputs (responses to a plurality of function evaluation items) by the subject via the input means 17. For example, the response inputs are responses by operations of a touch panel (a mouse) or the like, or voice inputs by voice input means (a microphone), and the like by the subject (step S11).

Then, based on the inputted responses, the evaluation means 11 evaluates the states of the ophthalmic functions, the neuro-ophthalmic functions and/or the cerebral nerve ophthalmic functions (the recovery states) of the subject (step S13). For the evaluations of the recovery states, it is preferable to use correlation data of the ophthalmic function evaluation items, the neuro-ophthalmic function evaluation items and/or the cerebral nerve ophthalmic function evaluation items.

Then, it is judged whether the grasped recovery states have reached a predetermined level (for example, a level at which the subject can spend his everyday life without trouble) or not (step S15), and the process is ended if the states have reached the predetermined level (in the case of “Yes” at the judgment). If it is judged that the states have not reached the predetermined level (in the case of “No” at the judgment), evaluation items thought to be required for the subject are extracted.

The program generation means 12 selects a program making it possible to perform training, focusing on a dysfunction (an item the criterion of which has not been reached) to generate a training program 103 again (step S07). In this case also, though only an item corresponding to a dysfunction may be extracted, it is desirable to select and combine a plurality of evaluation-item-specific programs not only for a single item but for items including those corresponding to normal functions and those corresponding to dysfunctions, because function evaluation items are often related to one another, and such relationships are not clear for some items. After that, evaluations by the evaluation means 11 and generation of a training program 103 by the program generation means 12 are repeated until the subject's functions reach the predetermined level.

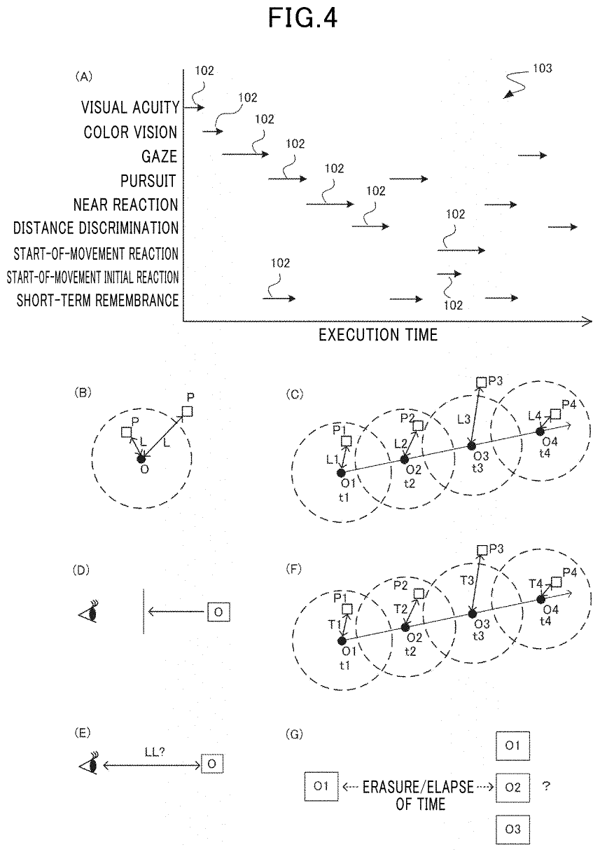

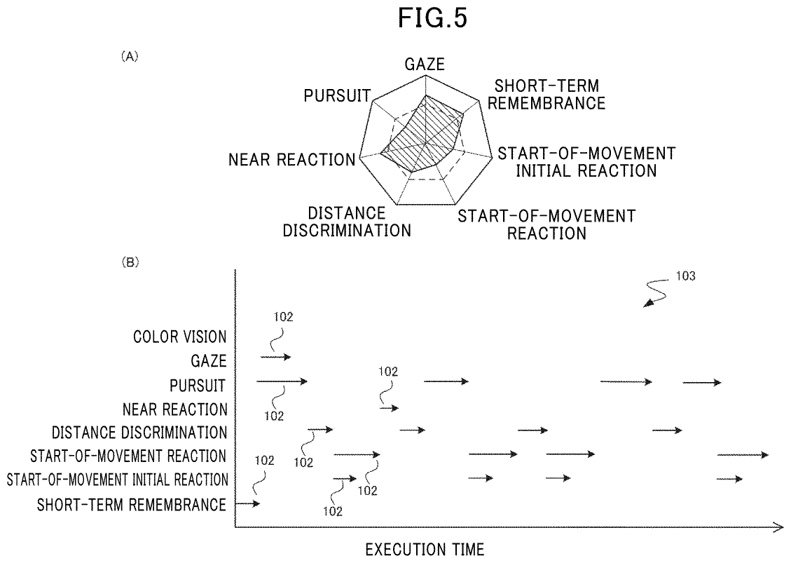

FIGS. 4 and 5 are overview diagrams illustrating examples of the function recovery training system 10 more specifically. FIG. 4(A) is an overview diagram showing a flow of function evaluation by the standard program 101, and FIGS. 4(B) to 4(F) show examples of display of the function evaluation items and an evaluation method. FIG. 5(A) shows an example of evaluation results, and FIG. 5(B) is an overview diagram showing a flow of function evaluation by the training program 103.

As shown in FIG. 4(A), the standard program 101 evaluates (measures) visual acuity, color vision, eyeball movement/eyeball movement adjustment function, and the like as ophthalmic functions, and evaluates gaze, pursuit, near reaction, distance discrimination (stereopsis), start-of-movement reaction, start-of-movement initial reaction, short-term remembrance and the like as neuro-ophthalmic functions (see the minor classifications in FIG. 2).

Each of the functions is evaluated based on execution time corresponding to the length of an arrow from the left to the right of the figure accompanying the elapse of time. That is, in this example, in order of visual acuity, color vision, gaze, pursuit, near reaction, distance discrimination . . . , programs for evaluating these functions are executed. Further, for example, the first evaluations of gaze and short-time remembrance are overlappedly evaluated (for example, by common display).

As for the evaluations of visual acuity and color vision, a method similar to known evaluation (measurement) used at ophthalmology clinics is used. Specifically, Landolt rings used in the case of visual acuity; and, for example, an Ishihara color blindness test chart, Standard Pseudoisochromatic Plates (SPP) or the like used in the case of color vision are displayed on the output means (for example, a display) 16, and the subject is prompted to answer. The evaluations are made by known methods for visual acuity and color vision, respectively.

As for the evaluation of eyeball movement/eyeball movement adjustment function, for example, a moving aim object (target) is displayed on the display means 16, and the subject is successively prompted to follow it with a pointer (a cursor) without delay though it is not shown in the figure. Further, the target is stopped at a predetermined position, and the subject is prompted to place the pointer on the target for a certain period of time. When the certain period of time elapses in the state in which the pointer is placed on the target, one point is fixed. The subject is caused to do this with his right or left eye and then caused to do it with both eyes. Thereby, a visual angle deviation, a pursuit movement speed and a deviation distance of each eye and of both eyes are read, and ophthalmic functions such as eyeball movement/eyeball movement adjustment function can be judged therefrom.

FIG. 4(B) is an overview diagram illustrating the evaluation of gaze. In the evaluation of gaze, for example, an aim object (a target) O is displayed on the output means 16, and the subject is prompted to perform an operation (a response) of placing an indicator (a pointer) P on the aim object O. An evaluation result is quantitatively obtained, for example, as an amount of deviation L of the pointer P from the target O or obtained by giving a score corresponding to whether the pointer P is within a predetermined distance (indicated by a broken line) from the target O or not.

FIG. 4(C) is an overview diagram illustrating the evaluation of pursuit. In the evaluation of pursuit, for example, a moving aim object (target) O1 to O4 is displayed on the output means 16, and the subject is prompted to perform an operation (a response) of placing a corresponding indicator (pointer) P1 to P4 on the aim object O1 to O4 each time the aim object O1 to O4 moves. An evaluation result is quantitatively obtained, for example, as an amount of deviation L1 (the same for L2 to L4) of the pointer P1 (the same for P2 to P4) from the target O1 (the same for O2 to O4) or obtained by giving a score corresponding to whether the pointer P1 (the same for P2 to P4) is within a predetermined distance (indicated by broken lines) from the target O1 (the same for O2 to O4) or not, whether the pointer P1 to P4 can be placed at all positions of the moving target O1 to O4, or the like.

FIG. 4(D) is an overview diagram illustrating the evaluation of near reaction. In the evaluation of near reaction, for example, a target O that moves approaching the subject from afar is displayed on the display means 16, and the subject is prompted to perform an operation of the operation means (a touch panel, a keyboard or the like) when the target O moves to a predetermined position. Or alternatively, a target (a number, a symbol or the like) that moves approaching the subject from afar is displayed, and the subject is prompted to read the target at a predetermined position. An evaluation result is quantitatively obtained by giving a score corresponding to whether the operation could be performed at the predetermined position or not, or whether the target was correctly read or not.

FIG. 4(E) is an overview diagram illustrating the evaluation of distance discrimination. In the evaluation of distance discrimination, for example, a target O that looks like being located far away is displayed on the display means 16, and the subject is prompted to input a distance (reduced scale) LL (for example, from a reference position). An evaluation result is quantitatively obtained by giving a score corresponding to an error from a correct distance (reduced scale).

The evaluations of near reaction and distance discrimination can be used for the evaluation of stereopsis by using a three-dimensional display and adopting software compatible with the three-dimensional display for displaying a three-dimensional image.

As shown in FIG. 4(F), in the evaluation of start-of-movement reaction, for example, a moving aim object (target) O1 to O4 is displayed on the display 16 similarly to the case of pursuit, and the subject is prompted to instantaneously perform an operation (a response) of placing an indicator (a pointer) P1 to P4 on the aim object O1 to O4 each time the aim object O1 to O4 moves. An evaluation result is quantitatively obtained. For example, the evaluation result is obtained as times Ti to T4 required to click the pointer P1 to P4 after the target O1 to O4 is displayed, and time lags between the displays and the clicks, or obtained by giving a score corresponding to whether the pointer P1 to P4 is within a predetermined distance (indicated by broken lines) from the target O1 to O4 or not, whether the pointer P1 to P4 can be placed at all positions of the moving target O1 to O4, respectively, or the like.

In the evaluation of start-of-movement initial reaction, for example, a reaction to movement of the first movement of the target O1 among successive movements of the target in the start-of-movement reaction described above (a reaction by the time when the subject moves the pointer P1 after the target O1 that is in a state in which the timing of starting to move is unknown is displayed) is evaluated. An evaluation result is quantitatively obtained similarly to the case of the evaluation of start-of-movement reaction.

As shown in FIG. 4(G), in the evaluation of short-term remembrance, for example, a certain target (a character, a number, an image of an article for daily use, or the like) O1 is displayed on the display 16, and then erased after causing the subject to remember the position and the attribute. After elapse of a predetermined time, the subject is prompted to select the erased target (display the subject remembers) from among a plurality of displayed targets O1 to O3. An evaluation result is quantitatively obtained by giving a score corresponding to whether the subject could select the same display as the first display or not.

With reference to FIG. 4(A) again, the standard program 101 is configured to include all the above evaluations. For example, with elapse of time of the standard program 101, displays evaluating visual acuity/color vision, gaze, pursuit, near reaction/distance discrimination (stereopsis), start-of-movement reaction, start-of-movement initial reaction and short-term remembrance are sequentially or randomly given in succession (repeatedly). These evaluation displays may be individually given. For example, after a display for gaze, another display for near reaction may be given, or an evaluation display may be compositely given. As the composite evaluation display, for example, a display is given in which display of causing the subject to gaze/pursue a target, display of causing the subject to recognize an approaching display (near reaction/distance discrimination) and the like are mixed among a series of displays like a moving scene around the subject.

As shown in FIG. 5(A), the evaluations of the functions are comprehensively and multifacetedly displayed (outputted) on the terminal of the subject or the medical worker based on given scores. It is desirable to make an output in a manner that the evaluations are easy to visually grasp, for example, using a radar chart, a pie chart, a bar graph or the like.

In this example, as for gaze, near reaction and short-term remembrance, the subject has reached the criteria (indicated by a broken line). On the other hand, as for pursuit, distance discrimination, start-of-movement reaction and start-of-movement initial reaction, results show that the criteria have not been reached. In this case, the evaluation means 11 evaluates the functions of pursuit, distance discrimination, start-of-movement reaction and start-of-movement initial reaction as dysfunctions.

In this example, as the evaluation-item-specific programs 102, programs capable of making evaluations individually for (focusing on) the functions of visual acuity/color vision, gaze, pursuit, near reaction/distance discrimination (stereopsis), start-of-movement reaction, start-of-movement initial reaction and short-term remembrance, respectively, are prepared.

Then, the program generation means 12 judges that, for each of function evaluation items with low evaluations (pursuit, distance discrimination, start-of-movement reaction and start-of-movement initial reaction), a dysfunction has occurred, based on the evaluation results (the criterial states) shown in FIG. 5(A) and appropriately selects evaluation-item-specific programs 102 for recovering the functions, that is, evaluation-item-specific programs 102 at least for pursuit, distance discrimination, start-of-movement reaction and start-of-movement initial reaction to generate a training program 103.

In this case also, it is recommended to appropriately include evaluation-item-specific programs 102 for functions that are not insufficient, for example, functions of gaze and the like, with evaluation-item-specific programs 102 for functions judged to be insufficient as main programs.

FIG. 5(B) shows an overview of the training program 103 generated as a result. In comparison with the standard program, the training program 103 is configured so that training can be performed, focusing on function evaluation items with low evaluations (pursuit, distance discrimination, start-of-movement reaction and start-of-movement initial reaction). The program generation means 12 can change execution times of the selected evaluation-item-specific programs 102 (an execution ratio) and order of the programs (lengths and order indicated by arrows in FIG. 5(B)). Further, prior to or after generation of the training program 103, the execution times and execution ratio of the evaluation-item-specific programs or selectable evaluation-item-specific programs 102 may be presented (displayed on the display 16) as a menu so that the subject (or the medical worker) can make a selection (a change)

As shown in FIG. 5(B), by generating a training program 103 that multifacetedly/compositely includes other functions judged to be normal (for example, gaze, near reaction and short-term remembrance), focusing on functions evaluated to be insufficient (in this example, pursuit, distance discrimination, start-of-movement reaction and start-of-movement initial reaction), it may be possible to, even in the case of a dysfunction accompanied by a complicated relationship among functions that has not been cleared be effectively recovered.

Evaluations (the recovery states) of the training program 103 are also quantitatively shown similarly to FIG. 5(A). Then, based on the evaluations (the recovery states), necessary evaluation-item-specific programs 102 are selected again to generate the next training program 103. In this case also, it is recommended to appropriately include evaluation-item-specific programs 102 for other functions which are not insufficient, focusing on functions judged to be insufficient.

Then, by evaluations (of the recovery states) by the training program 103, and regeneration and re-execution of a training program 103 based thereon, training is repeated. In the case where the training program 103 is repeated a plurality of times, the degree of difficulty (a burden on the subject) is adapted to increase step by step according to the recovery states.

If correlation data exists, the correlation data is taken into consideration, as already stated in the description of the evaluations of (giving of scores to) the criterial states and the recovery states. For example, when the evaluation (the score) of visual acuity is improved as a result of training, it is conceivable that “it has become easier to see” merely because ocular muscles have been strengthened, and, therefore, there is a possibility that, for example, for gaze, pursuit and the like, the functions are also improved. In such a case, the evaluation (the score) corresponding to the improvement of visual acuity may be added to the evaluations (the scores) of gaze, pursuit and the like, or the evaluation (the score) corresponding to the improvement may be excluded, depending on cases.

Thus, the present function recovery training system 10 is not intended to clearly identify a cause and a part of a dysfunction, spending much time and effort, and cause such training that recovers only insufficient functions to be performed but to cause a plurality of functions to be compositely trained, focusing on functions judged to be insufficient. Thereby, it is aimed to, without deeply pursuing complicated relationships among functions, recover insufficient functions consequently. As a result, it is possible to reduce a burden on training of the subject, and it is possible for the subject to repeatedly perform the training alone. It is also possible for the medical worker and the like to intervene if necessary.

In this example, for example, measurement of visual acuity/color vision, which are ophthalmic functions, may be separately performed with known measurement devices dedicated to the functions. Further, in addition to execution of the standard program 101, for example, functions that are not included therein, such as refraction/adjustment may be separately measured with known measurement devices dedicated to the functions (for example, corneal shape/refraction analysis devices).

Evaluation (measurement) results obtained by separate devices (including the medical worker terminal device C2) and methods are captured via the intervention information processing means 14 as intervention information and can be reflected on the evaluations of the evaluation means 11 (the evaluations of the criterial states and the recovery states).

Further, though evaluation items of neuro-ophthalmic functions are omitted in this example, they may be included.

### EXAMPLE

A specific example of the function recovery training system 10 of the present embodiment will be explained below as one example.

It is recommended to incorporate the standard program 101 and the training program into a game to make it possible for the subject to repeatedly perform execution, enjoying the programs.

### Example 1

When the subject executes the function recovery training system 10 introduced in the subject terminal device C1 (for example, a smartphone), a bird forest spreads on the display means 16 by the standard program 101 (or the training program 103). A plurality of different birds come flying into the forest in turn and move to suitably find and enter nests. A bird as a target (configured so that the subject recognizes it as a target) is displayed and moved to enter a nest. The subject causes a bird as a pointer (a cursor) to move and causes the bird to enter the nest following the target bird that has entered the nest. Since the target bird flies around and stops at an arbitrary position, the subject pursues and follows the target bird until the target bird stops, and places the pointer bird on the target bird when the target bird stops.

By the series of operations (responses), cognitive behavior delay time for the start of movement of the pointer, a movement speed and the like are collected as a distance on the coordinate axes and clock time. Based on the collected results and accuracy, a score as an evaluation result is given.

Based on the evaluation result, necessary evaluation-item-specific programs are selected, and a training program 103 is generated. Further, the evaluation result is transmitted to the medical worker terminal device C2.

The training program 103 may be generated by the function recovery training system 10 in the subject terminal device C1 or may be generated by the function recovery training system 10 in the medical worker terminal device C2 and transmitted to the function recovery training system 10 in the subject terminal device C1 after the intervention processing (for example, alteration of a program, addition of data, and the like) by the medical worker is performed as necessary.

The subject repeatedly uses the training program 103 generated by the function recovery training system 10 in the subject terminal device C1 or transmitted from the medical worker terminal device C2 to perform training.

### Example 2

When the subject executes the function recovery training system 10 introduced in the subject terminal device C1 (for example, a smartphone), a scene that may be seen at the time of riding a roller coaster is displayed on the display means 16 by the standard program 101 (or the training program 103). The roller coaster goes on a straight course, and a plate on which a number is written is arranged in the middle of the course (it is visually recognized as if the plate were approaching). The subject remembers the size and color of the plate and the number, and inputs the size, the color and the number after a predetermined time elapses after the plate passes.

When (judging that) the plate approaches a predetermined position set by the standard program (or the training program), the subject operates the operation means (for example, taps a predetermined button on the touch panel). Evaluation is made according to a distance between the input by the subject and the predetermined position set by the program. In this case, evaluations of functions of short-term remembrance of remembering the plate, and near reaction can be made.

In this case, images of near reactions such as convergence and divergence of the ocular muscles and contraction of the ciliary body are picked up by a camera attached to the eyeball/pupil evaluation recording device (means) 32 and converted to data.

Further, in this case, evaluation of stereopsis may be made using a 3D image. Since stereopsis is influenced by the magnitude of a difference between left and right monocular ophthalmic functions, measurement of the monocular ophthalmic functions is performed, for example, by the standard program 101 in advance so that the training program 103 may be generated based on a result of the measurement.

### Example 3

The function recovery training system 10 can be used for diagnosis of dementia for a senior driver's license test and the like.

Evaluations of convergence and short-term remembrance (long-term remembrance) becomes possible by causing the subject to recognize a change in flashing among many lit signals and prompting the subject to respond or by causing the subject to remember the number and color of a suddenly approaching target (car) and notify the number after a while.

Modifications and additional components of the present embodiment will be enumerated below. The components below can be implemented, by being appropriately combined or exchanged with those of the embodiment described above.

Further, there may be provided information exchange means for associating the subject with information about the subject (for example, subject ID information, the subject's ophthalmic functions, neuro-ophthalmic functions and cerebral nerve ophthalmic functions, the standard program 101, the evaluation-item-specific programs 102, the training program 103 and the like) and transmitting the information from one function recovery training system to an external device (another function recovery training system) as data to cause the other function recovery training system to function in synchronization with the one function recovery training system.

Further, the intervention information (especially intervention information transmitted from the medical worker terminal device C2) may include, for example, medical record information and the like at the time of the subject having visited a medical institution in advance.

Further, there may be provided with means for, when the subject is executing the training program 103 alone, recommending consultation at a medical institution as necessary.

### Example 4

By using the function recovery training system 10 of the present embodiment, there may be a case where, when a part of brain functions (especially a part related to the vision) are insufficient, function recovery can be expected by performing effective training. Specifically, there may be a case where it is possible to easily and efficiently perform training for a part of dysfunctions because of dementia.

As an example, evaluation results of a Hasegawa simple intelligence evaluation scale, which is a diagnostic method for dementia, are read as intervention information via the intervention information processing means 14, and the criterial states are evaluated based on the standard program 101 considering the intervention information. Then, a necessary training program 103 is generated based on the criterial states, and training is performed.

Or alternatively, the criterial states are evaluated based on an ordinary standard program 101 (in a state of not using intervention information). Then, the evaluation results of the Hasegawa simple intelligence evaluation scale are read as intervention information via the intervention information processing means 14; a necessary training program 103 is generated based on the criterial states and the intervention information; and training is performed.

Then, after the training program 103 is used for a certain period, evaluation is made with a Hasegawa simple intelligence evaluation scale again, and a result of the evaluation is read as intervention information via the intervention information processing means 14. By comparing evaluation results (recovery states) of the latest training program 103 and new intervention information, by the evaluation means 11, correlation between the evaluation results of the Hasegawa simple intelligence evaluation scale and the function recovery training system 10 of the present embodiment can be grasped.

By holding the correlation relationship as correlation data and generating a training program 103 considering the correlation data, it is possible to utilize the training program 103 for dementia rehabilitation.

An embodiment of the present invention has been explained above. Dementia is not a single disease name but a diagnosed name corresponding to a combination of many elements. In most cases, a patient group and a normal group are judged using physical phenomena brought about as a result of brain activity, which are thought to be easy to measure and analyze, such as brain waves and a brain magnetic field and a cerebral circulation blood flow. That is, it can be said that, even if such a physical phenomenon appears, it is difficult to identify the place thereof or grasp a correspondence relationship about which of minute brain cells has a trouble. It is difficult that this leads to solution/relief of dementia itself or of a patient's problems accompanying the dementia, and, in the actual situation, effective and reasonable rehabilitation means for relieving symptoms of dementia has not been developed.

In order to evaluate cerebral nerve functions in diagnosis of dementia or the like, it is sufficient if sensory organs of the five senses normally react to reaction from outside, and the subject can behave. It can be said that, in the case of a human being, it is sufficient if higher functions such as remembrance and judgment, which are highly developed cerebral functions, are additionally at a normal level.

According to the invention of the present application, even in a case where brain functions (functions of cerebral nerves) are insufficient, such as dementia, it is possible to relieve dysfunction symptoms (for example, dementia) and recover the symptoms not by diagnosing or treating the insufficient brain functions as a certain disease name but by accurately and efficiently evaluating the neuro-ophthalmic functions and the cerebral nerve ophthalmic functions and performing training therefor. That is, the function recovery training system 10 of the present embodiment can be also applied to rehabilitation means for dementia.

Thus, the function recovery training system 10 of the present embodiment provides a method that is easy to repeat to the subject, the method being for recovering normal functions within a reasonable range. A standard program and a training program are presented to the subject to recover the neuro-ophthalmic functions and, at the same time, promote recovery of the cerebral nerve functions closely related to the neuro-ophthalmic functions.

The cerebral nerve functions the recovery of which is prompted by implementation of the function recovery training system 10 of the present embodiment are thought to be those that are closely related to the ophthalmic functions and the neuro-ophthalmic functions, among higher brain functions such as remembrance and conjecture. However, by nerve stimulation due to appearance of the ophthalmic and neuro-ophthalmic functions repeatedly reaching the cerebral nerves, it is also expected that function recovery or function substitution by other brain cells may occur.

Though the explanation has been made, with “the vision” as an example of a biological function in the embodiment described above, the embodiment can be similarly applied to other perceptions such as smell, taste, hearing and touch.

The function recovery training system 10 of the present embodiment has been explained above. A function recovery training device that integrally accommodates the functions of the above function recovery training system 10 is also possible, and a program capable of causing a computer to operate as the function recovery training system 10 or the function recovery training device is also possible.

That is, the present invention is a function recovery training device for performing training to recover biological functions of a subject, the function recovery training device including: evaluation means 11; program generation means 12; storage means 13; input means 17; and output means 16; wherein the storage means 13 may be means for holding: a standard program 101 capable of evaluating states of the functions to be evaluation criteria based on a plurality of function evaluation items; and evaluation-item-specific programs 102 making it possible to perform training individually for each of the plurality of function evaluation items; the evaluation means 11 may be means for executing the standard program 101 to output a predetermined target to the output means 16, accepting a response to the target from the subject via the input means 17, evaluating criterial states of the functions based on the response; executing the training program 103 to output a predetermined target to the output means 16, accepting a response to the target from the subject via the input means 17, and evaluating recovery states of the functions based on the response; and the program generation means 12 may be means for selecting any of the evaluation-item-specific programs 102 that is for at least one of the plurality of function evaluation items based on the evaluated criterial states to generate the training program 103.

Further, the present invention is a program including a standard program 101 and evaluation-item-specific programs 102, the program causing a computer to execute a recovery training process for biological functions, wherein the standard program 101 is configured to be capable of evaluating states of the functions to be evaluation criteria based on a plurality of function evaluation items; the evaluation-item-specific programs 102 are configured to make it possible to individually perform training for the plurality of function evaluation items, respectively; and the program may cause the computer to execute the steps of: executing the standard program 101 and evaluating criterial states of the functions based on a response from a subject; selecting any of the evaluation-item-specific programs 102 that is for at least one of the plurality of function evaluation items based on the evaluated criterial states to generate a training program 103; executing the training program 103 and evaluating recovery states of the functions based on a response from the subject.

The function recovery training system 10, the function recovery training device and the program of the present invention are not limited to the embodiment described above, and it is, of course, possible to make various kinds of changes within a range not departing from the spirit of the present invention.

### REFERENCE SIGNS LIST

10 Function recovery training system
11 Evaluation means
12 Program generation means
13 Storage means
14 Intervention information processing means
15 Control means
16 Output means (display means)
17 Input means
19 Communication means
21 Evaluation means
22 Program generation means
23 Storage means
24 Intervention means
25 Control means
26 Output means
27 Input means
29 Communication means
30 External device
31 Field-of-view adjustment switching device
32 Eyeball/pupil evaluation recording device
33 Eyeball image pickup device
101 Standard program
102 Programs by evaluation item
103 Training program
C1 Subject terminal device
C2 Medical worker terminal device

1. A function recovery training system for performing training to recover biological functions of a subject, the function recovery training system comprising a terminal device, the terminal device comprising:
evaluation means;
program generation means;
storage means;
input means; and
output means, wherein
the storage means holds:
a standard program capable of evaluating states of the functions to be evaluation criteria based on a plurality of function evaluation items; and
evaluation-item-specific programs making it possible to perform training individually for each of the plurality of function evaluation items;
wherein the evaluation means outputs a predetermined target to the output means by execution of the standard program, accepts a response to the target from the subject via the input means, and evaluates criterial states of the functions based on the response;
wherein the program generation means selects any of the evaluation-item-specific programs that is for at least one of the plurality of function evaluation items based on the evaluated criterial states to generate a training program; and
wherein the evaluation means outputs a predetermined target to the output means by execution of the training program, accepts a response to the target from the subject via the input means, and evaluates recovery states of the functions based on the response.

2. The function recovery training system according to claim 1, wherein
the program generation means generates the training program by combining two or more of the evaluation-item-specific programs selected based on the criterial states.

3. The function recovery training system according to claim 1, wherein
the functions are functions related to vision.

4. The function recovery training system according to claim 1, wherein
the function evaluation items include an item related to neuro-ophthalmic functions (hereinafter referred to as “the neuro-ophthalmic function evaluation item”).

5. The function recovery training system according to claim 4, wherein
the function evaluation items include an item related to ophthalmic functions (hereinafter referred to as “the ophthalmic function evaluation item”).

6. The function recovery training system according to claim 5, wherein
the evaluation means evaluates the criterial states based on correlation between the neuro-ophthalmic function evaluation item and the ophthalmic function evaluation item.

7. The function recovery training system according to claim 5, wherein
the evaluation means evaluates the recovery states based on correlation between the neuro-ophthalmic function evaluation item and the ophthalmic function evaluation item.

8. The function recovery training system according to claim 1, comprising:
output means for outputting a predetermined target at least based on execution of the standard program and the training program; and
input means capable of inputting a response of the subject to the target.

9. The function recovery training system according to claim 1, comprising:
intervention information processing means capable of processing intervention information inputted from outside.

10. The function recovery training system according to claim 9, comprising:
another terminal device capable of transmitting the intervention information; and
a communication line connecting the terminal device and the other terminal device.

11. A function recovery training device for performing training to recover biological functions of a subject, the function recovery training device comprising:
evaluation means;
program generation means;
storage means;
input means; and
output means, wherein
the storage means is means for holding:
a standard program capable of evaluating states of the functions to be evaluation criteria based on a plurality of function evaluation items; and
evaluation-item-specific programs making it possible to perform training individually for each of the plurality of function evaluation items;
wherein the evaluation means is means for outputting a predetermined target to the output means by execution of the standard program, accepting a response to the target from the subject via the input means, evaluating criterial states of the functions based on the response; outputting a predetermined target to the output means by execution of a training program, accepting a response to the target from the subject via the input means, and evaluating recovery states of the functions based on the response; and
wherein the program generation means selects any of the evaluation-item-specific programs that is for at least one of the plurality of function evaluation items based on the evaluated criterial states to generate the training program.

12. A program comprising a standard program and evaluation-item-specific programs, the program causing a computer to execute a recovery training process for biological functions, wherein
the standard program is configured to be capable of evaluating states of the functions to be evaluation criteria based on a plurality of function evaluation items; and
wherein the evaluation-item-specific programs are configured to make it possible to individually perform training for the plurality of function evaluation items, respectively; and
wherein the evaluation-item-specific programs cause the computer to execute the steps of:
executing the standard program to output a predetermined target to a subject;
accepting a response to the target from the subject;
evaluating criterial states of the functions based on the response;
selecting any of the evaluation-item-specific programs that is for at least one of the plurality of function evaluation items based on the evaluated criterial states to generate a training program;
executing the training program to output a predetermined target to the subject;
accepting a response to the target from the subject; and
evaluating recovery states of the functions based on the response.
